(19) United States
(12) Patent Application Publication
(10) Pub. No.: US 2005/0123484 A1
(43) Pub. Date: Jun. 9, 2005

(54) Non-flammable topical anesthetic liquid aerosols

(75) Inventors: Jane Hirsh, Wellesley, MA (US); Donald L. Tibbetts, North Falmouth, MA (US); Mark Hirsh, Wellesley, MA (US)

Correspondence Address: PATREA L. PABST;PABST PATENT GROUP LLP, 400 COLONY SQUARE, SUITE 1200, ATLANTA, GA, 30361, US

(73) Assignee: Collegium Pharmaceutical, Inc.

(21) Appl. No.: 10/956,819

(22) Filed: Oct. 1, 2004

Related U.S. Application Data
(60) Provisional application No. 60/508,186, filed on Oct. 2, 2003.
(60) Provisional application No. 60/560,890, filed on Apr. 9, 2004.

(51) Int. Cl. A61L 9/04; A61K 31/24

(52) U.S. Cl. 424/45; 514/535

(57) ABSTRACT

A topical liquid aerosol formulation for accurate metered dose delivery has been developed which includes a concentrate comprising a local anesthetic in a non-alcohol solvent and a hydrofluorocarbon (HFC) propellant. In the preferred embodiment, the concentration of the non-alcohol solvent in the concentrate is between about 75% and 85% by weight of the formulation. In the most preferred embodiment, the non-alcohol solvent is a water-soluble polyol such as ethylene glycol, propylene glycol, glycerol, diethylene glycol, dipropylene glycol, oligoalkylene glycols, liquid polyalkylene glycols, or combinations thereof. In one embodiment, the concentration of the local anesthetic in the concentrate is between about 15% and 25% by weight. In the preferred embodiment, the hydrofluorocarbon propellant is 1,1,1,2-tetrafluoroethane 1,1,1,2,3,3,3-heptafluoropropane or combinations thereof, in a concentration between about 35% and 65% by weight of the final formulation, more preferably between about 45% and 55% by weight of the final formulation. A particularly preferred formulation includes benzocaine, tetracaine, and butylaminobenzoate, wherein the concentration of benzocaine in the concentrate is 14% by weight, the concentration of tetracaine in the concentrate is 2% by weight, and the concentration of butylaminobenzoate in the concentrate is 2% by weight. It has been found that the formulation is more stable in the substantial absence of oxygen. The formulation is preferably administered using a metered dose device for release of a controlled amount of the local anesthetic.

### CROSS-REFERENCE TO RELATED APPLICATIONS

This application claims benefit of U.S. Provisional Application Nos. 60/508,186, entitled “Non-Flammable Topical Anesthetic Aerosol Spray”, filed Oct. 2, 2003, by Mark Hirsh, and 60/560,890, entitled “Non-Flammable Topical Aerosol Spray”, filed Apr. 9, 2004 by Jane Hirsh and Donald L. Tibbetts.

### BACKGROUND OF THE INVENTION

The present invention is generally in the field of liquid aerosols, especially for topical delivery of local anesthetics.

The use of chlorofluorocarbons as aerosols and refrigerants was banned under the 1987 Montreal Agreement and the production of these propellants was restricted worldwide beginning in 1989. Certain pharmaceutical aerosols for inhalation that use fluorotrichloromethane (CFC-11), difluorodichloromethane (CFC-12) and dichlorotetrafluoroethane (CFC-114) as propellants were exempted from the ban. These propellants can still be used for aerosol formulations for inhalation if they were grandfathered in under the 1987 agreement. However, new or revised aerosol formulations may not contain CFC propellants, and alternative propellants must be used that are more environmentally friendly. Therefore, manufacturers must reformulate or modify existing products to use non-CFC propellants, while maintaining important aspects of the previous formulation, such as accuracy of delivery, stability, etc.

Providing reproducible performance of reformulated non-CFC aerosols for pharmaceutical uses represents a challenging task. Users of propellants intended for pulmonary drug delivery have generally tried to reformulate with approved alternate hydrofluorocarbons (HFCs, also known as HIFAs and hydrofluoroalkanes) and cosolvents such as ethanol, since the lower solvating power of the HFC propellants compared to CFCs is not readily overcome. Alternatively, manufacturers have used volatile hydrocarbons such as n-butane, propane and isobutene, together with cosolvents such as ethanol, as the propellant. Examples include “Hurricane”® spray (Beutlich L.P. Pharmaceuticals, Waukegan, Ill.) and “Topex”®20% benzocaine spray (Sultan Dental Products, Engelwood, N.J.), both of which use volatile hydrocarbons (butane, propane, etc.) and co-solvents such as ethanol. It is believed that only one topical anesthetic spray, “Cetacaine”™ spray (Cetylite Industries Inc., Pennsauken, N.J.), a non-metered spray containing a combination of benzocaine, butyl aminobenzoate and tetracaine, still uses chlorofluorocarbon propellants as a grandfathered use under the Montreal Agreement.

Reformulation of propellants is difficult, and normally requires re-approval of the formulation:

“[S]ince replacing a component of any formulation means introducing new properties and characteristics, there are significant challenges in that there is no analog or direct replacement for CFC-11 and its associated solvency. This had led some formulators to use ethanol.” (Pharmaceutical Aerosols, Jun. 2003 pg. 21).

Ethanol, however, at concentrations of about 20% or more, is a drying agent and irritatant. Moreover, ethanol and other lower alcohols are quite volatile, and are a fire and explosion hazard during both manufacture and use. The addition of volatile alkanes as propellants further increases the fire and explosion hazard of the aerosol propellant. Hence, replacing CFC propellants is not a simple matter of substitution of a HFC for a CFC. Currently, there are no known substitutes for CFCs that do not require the use of potentially hazardous cosolvents and co-propellants. At most, the problem has been solved for a few materials that are actually soluble in the HFCs, such as prilocaine and lidocaine, or by the use of clathrates and surfactant-treated solids as delivery means.

For example, U.S. Pat. No. 5,858,331 to Henry describes an aerosol formulation containing prilocaine base which is soluble in a hydrofluorocarbon propellant without the addition of a cosolvent. U.S. Pat. No. 5,593,661 to Henry describes a topical aerosol formulation containing the local anesthetic lidocaine, in free base form, dissolved in a hydrofluorocarbon propellant without the addition of a cosolvent. These formulations, however, are limited to those local anesthetics which are soluble in hydrofluorocarbons.

An additional problem with many spray products is accuracy of delivery. For example, the Hurricane product described above is a continuous spray, and the directions state, “Spray ½ second. Repeat if necessary.” Likewise, users of CETACAINE™ are instructed to spray for “approximately one second”. TOPEX™, a metered spray, states that a single metered dose dispenses 50 mg of the topical solution, equivalent to 10 mg. of delivered benzocaine. However, when a sample was tested, it was found that delivery was significantly lower than stated (25 mg rather than 40 mg in four spray doses.)

Yet another problem with some current formulations is degradation of the product during packaging. Certain combinations of multiple anesthetics can have stability problems resulting in degradation, lack of potency, and recalls (e.g., FDA Enforcement reports of Jan. 10, 1996 and Feb. 28, 1996, recalling lots of CETACAINE™ spray anesthetic).

It is therefore an object of the invention to provide topical anesthetic liquid aerosol spray formulations that use environmentally friendly HFCs as the propellant without the need for flammable cosolvents, such as volatile alcohols or flammable alkanes, such as propane and butane, as co-propellants.

It is another object of the invention to provide topical anesthetic liquid aerosol spray formulations for metered dose delivery in order to avoid adverse side effects.

It is yet another object of the invention to provide topical anesthetic liquid aerosol spray formulations which are stable over an extended period of time.

### BRIEF SUMMARY OF THE INVENTION

A topical liquid aerosol formulation for accurate metered dose delivery has been developed which includes a concentrate comprising a local anesthetic in a non-alcohol solvent and a hydrofluorocarbon (HFC) propellant. In the preferred embodiment, the concentration of the non-alcohol solvent in the concentrate is between about 75% and 85% by weight of the formulation. In the most preferred embodiment, the non-alcohol solvent is a water-soluble polyol such as ethylene glycol, propylene glycol, glycerol, diethylene glycol, dipropylene glycol, oligoalkylene glycols, liquid polyalkylene glycols, or combinations thereof. In one embodiment, the concentration of the local anesthetic in the concentrate is between about 15% and 25% by weight, and the local anesthetic is lidocaine, prilocaine, bupivacaine, levo-bupivacaine, ropivacaine, mepivacaine, procaine, chloroprocaine, propoxycaine, hexylcaine, tetracaine, cyclomethycaine, benoxinate, butacaine, proparacaine, butamben, diperodon, phenacaine, falicaine, dyclonine, pramoxine, dimethisoquien, benzocaine, amethocaine, dibucaine, ketocaine, propanocaine, propipocaine, or combinations thereof. In the preferred embodiment, the concentration of any additional excipients in the concentrate is between about 0.5% and 3% by weight. In the preferred embodiment,the hydrofluorocarbon propellant is 1,1,1,2-tetrafluoroethane, 1,1,1,2,3,3,3-heptafluoropropane or combinations thereof, in a concentration between about 35% and 65% by weight of the final formulation, more preferably between about 45% and 55% by weight of the final formulation.

A particularly preferred formulation includes benzocaine, tetracaine, and butylaminobenzoate, wherein the concentration of benzocaine in the concentrate is 14% by weight, the concentration of tetracaine in the concentrate is 2% by weight, and the concentration of butylaminobenzoate (“butamben”) in the concentrate is 2% by weight.

It has been found that the formulation is more stable in the substantial absence of oxygen. The oxygen can be removed by purging the concentrate with an inert gas, cold filling the hydrofluorocarbon, preparing the formulation under vacuum and combinations thereof. Trace oxygen can be removed by antioxidants, such as BHT, BHA, vitamin E, and other pharmaceutically-acceptable antioxidants.

The formulation is preferably administered using a metered dose device for release of a controlled amount of the local anesthetic.

### DETAILED DESCRIPTION OF THE INVENTION

I. Compositions

a. Propellants

The gaseous propellant consists primarily of HFCs. Suitable propellants include HFCs such as 1,1,1,2-tetrafluoroethane (134a) and 1,1,1,2,3,3,3-heptafluoropropane (227), but mixtures and admixtures of these and other HFCs that are currently approved or may become approved for medical use are suitable. The propellants of the invention preferably exclude concentrations of hydrocarbon propellant gases, including particularly butanes, butenes, and propane, which are sufficient to produce flammable or explosive vapors during spraying. Furthermore, the aerosol spray has a limited concentration of volatile alcohols, including particularly ethanol, methanol, propanol and isopropanol, and butanols. The preferred limiting concentration in the mixture is, as with the gases, the concentration at which the sprayed material becomes flammable or explosive.

b. Solvents for Dissolution of the Local Anesthetic

The HFC contains a solvent, of relatively low vapor pressure, to dissolve the local anesthetic. Preferably, the vapor pressure of the solvent at atmospheric pressure and room temperature is less than its lower flammable limit. The solvent, which may be a single material or a mixture of more than one chemical species, preferably does not contain any volatile alcohols, particularly aliphatic and unsaturated alcohols having one to four carbons. The solvent must also be suitable for administering to the skin, to mucosal membranes, or to the respiratory tract, depending on the intended use of the preparation. A preferred class of solvents is the liquid polyols, i.e. molecules having two or more hydroxyl groups and being liquids at room temperature and atmospheric pressure. Examples of suitable polyols include ethylene glycol, propylene glycol, glycerol, diethylene glycol, dipropylene glycol, oligoalkylene glycols, liquid polyalkyleneglycols, and mixtures thereof. The oligo- and polyalkylene glycols are often liquids up to molecular weights in the range of 3000 to 5000 Daltons, although lower molecular weights will generally be preferred. Lower alkyl ethers of such polyols may also be suitable, provided they are liquids at room temperature and atmospheric pressure and they have been approved for medical use. In a preferred embodiment, the solvent is dipropylene glycol.

c. Local Anesthetics

Classes of local anesthetics which can be utilized include the aminoacylanilide compounds such as lidocaine, prilocaine, bupivacaine, levobupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine. The anesthetic can exist as the free-base form or a pharmaceutically acceptable salt.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

d. Excipients

Formulations may be prepared using a pharmaceutically acceptable excipient composed of materials that are considered safe and effective and may be administered to an individual without causing undesirable biological side effects or unwanted interactions. The excipient is all components present in the pharmaceutical formulation other than the active ingredient or ingredients. As generally used herein “excipient” includes, but is not limited to sweetening agents, flavorants and preservatives.

Flavorants can be synthetic or naturally occurring compounds. Suitable flavorants include, but are not limited to, anise oil, cinnamon oil, cocoa, menthol, orange oil, peppermint oil, and vanillin. Suitable sweetening agents include, but are not limited to, saccharin, aspartame, dextrose, glycerin, mannitol, sorbitol, and sucrose. In a preferred embodiment, saccharin is used as a sweetening agent.

Preservatives are used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal. Preservatives can also include antioxidants such a BHA, BHT, vitamin E, and other pharmacologically acceptable antioxidants. In a preferred embodiment, benzalkonium chloride and cetyldimethylammonium bromide are used as preservatives.

II. Method of Administration

a. Administration of the Formulation to a Patient

The aerosol spray is administered as a liquid for the administration to all accessible mucous membranes (excluding the eyes) to control pain, itching, and gagging.

b. Metered Dose Delivery

The aerosol is preferably administered in a metered device. This is important because the exclusion of lower alcohols may lead to higher pressures in the canister, since HFCs are typically less soluble in a glycol than they are in alcohols. Spray metering devices, which allow only a fixed volume of liquid to be delivered for each push of a button or equivalent act, are well known in the art and are easier for users to control. The spray can, with the metering device, will typically be a metal can, such as an aluminum can, and will usually be lined with an inert polymeric coating to prevent interaction of the metal with the medication. The dispensing device can alternatively be glass or plastic, but those are less preferred because of the higher pressures of the compositions of the invention.

Metered delivery is also important when delivering some topical anesthetics, particularly benzocaine, because overdoses of these anesthetics can cause methemoglobinemia. This is well documented in the literature (c.f. Novaro et al, Journal of the American Society of Echocardiography, vol. 16, no. 2, p. 170-175, 2003; Guertler et al, Fundamental and Applied Toxicology, Vol. 18, p 294-298, 1992; Khorasani et al (abstract in PubMed, PMID 11159236) Anesth. Analg. 2001 February; vol 92 no 2; pg. 379-83). The currently marketed continuous spray preparations (Cetacaine®, Hurricaine®) were found to be easy to overadminister, and thus to be possible causes of methemoglobinemia. Thus, a spray medicament containing benzocaine, or other medicaments potentially causing methemoglobinemia, preferably should be metered to provide reproducible dose delivery.

III. Method of Making the Formulation

a. Concentrate

A concentrate is prepared by dissolving the anesthetic in a non-alcohol solvent. Suitable excipients including sweetening agents such as saccharin and preservatives such as benzalkonium chloride and cetyldimethylammonium bromide can be added to this solution. Oxygen can be removed from the concentrate by bubbling an inert gas through the concentrate or by adding antioxidants such a BHA, BHT, vitamin E, and other pharmacologically acceptable antioxidants.

The concentration of the local anesthetic in the concentrate is typically 15% to 25% by weight. The concentration of the water soluble polyol in the concentrate is about 75% to about 85% by weight. The concentration of excipients, such as sweetening agents and preservatives, in the concentrate, if any, is from about 0.5 to about 3% by weight.

The concentrate is placed in a can, the can is sealed, and the HFC propellant is added. The weight of HFC propellant is in the range of about 35 to 65% of the final weight, more typically about 45% to 55%. In a preferred formulation, the concentrate contains about 14% benzocaine, about 2% butylaminobenzoate, and about 2% tetracaine, by weight.

b. Topical Liquid Aerosol for Metered Dose Delivery

A concentrate containing the local anesthetic and excipients, if any, dissolved in a non-alcohol solvent is added to a plastic-lined open aluminum can. A metered spray assembly, including a can lid and a dip tube, is installed and the joint between the lid and the can is crimped to form a pressure-tight seal between the lid and the can. The hydrofluorocarbon is added through the spray assembly. The reproducibility of delivery of the local anesthetic with the metered dose device is then determined. When deoxygenation is desired, it is preferably performed on the concentrate before addition of the propellant; the propellant can be deoxygenated separately if desired.

c. Stability of the Formulation

It was found that preparation of the formulation of Example 1 substantially free of oxygen, maintained by loading into aerosol cans in the absence of oxygen, prevented the loss of tetracaine on storage. It appears that a specific interaction of tetracaine with oxygen occurs in the presence of certain catalytic materials, which include benzocaine and butamben, which results in the loss of tetracaine and the appearance of a possible degradation product which is detected by chromatography. This unknown material appeared in the formulations in which tetracaine was degraded, eluting at about five minutes.

The usefulness of exclusion of air from aerosol preparations does not seem to be widely appreciated. The particular method of removing oxygen (air) was to bubble argon through the solution overnight while the anesthetics and excipients were dissolving in the dipropylene glycol, and then maintaining the solution in an argon-filled glove bag until it was bottled in the aerosol cans.

Industrial scale methods may include, without limitation, purging with less-expensive gases (such as nitrogen); using cold (liquid) HFCs during filling and allowing some excess gas to bleed, preferentially removing lower-boiling gases; and conduction of preparation processes under vacuum.

### EXAMPLES

### Example 1

A nonaqueous formulation for a spray aerosol topical anesthetic was prepared as a concentrate solution of non-gaseous ingredients.

The concentrate contained:

| Ingredient | Percent by weight |
|---|---|
| Dipropylene glycol | 80.6 |
| Benzocaine | 14.0 |
| Butamben | 2.0 |
| Tetracaine | 2.0 |
| Saccharin | 0.50 |
| Benzalkonium chloride | 0.555 |
| Cetydimethylammonium bromide | 0.005 |
| Flavor | 0.34 |
| TOTAL: | 100.0% |

### Example 2

An alternate nonaqueous formulation for a spray aerosol topical anesthetic was prepared as a concentrate solution of non-gaseous ingredients.

The concentrate contained:

| Ingredient | Percent by weight |
|---|---|
| Dipropylene glycol | 78.74 |
| Benzocaine | 20.0 |
| Saccharin | 0.50 |
| Benzalkonium chloride | 0.555 |
| Cetydimethylammonium bromide | 0.005 |
| Flavor | 0.20 |
| TOTAL: | 100.0% |

### Example 3

Packaging of Spray Aerosol topical anesthetic in the substantial absence of Oxygen.

32 grams of the concentrate of Example 2 was placed into a plastic-lined open aluminum can. A metered sprayer assembly, including a can lid and a dip tube, was installed, and the joint was crimped to form a pressure-tight seal between the lid and the can. Then 28 grams of HFA 134a propellant was added through the spray assembly.

35 grams of the concentrate of Example 1 was placed into a plastic-lined open aluminum can. A metered sprayer assembly, including a can lid and a dip tube, was installed, and the joint was crimped to form a pressure-tight seal between the lid and the can. Then 25 grams of HFA 134a propellant was added through the spray assembly.

### Example 4

Measurement of Accuracy of Metered Delivery

The reproducibility of delivery of material with a spray can loaded as described in Example 3 was determined. 10 doses were dispensed into separate flasks. The dispensed material is taken up in an appropriate solvent, and the amounts of benzocaine, butamben and tetracaine were determined by HPLC. The coefficient of variation (the standard deviation divided by the average) of deposition was found to be less than 5% for each of the three anesthetic components. In contrast, attempts to spray an identical quantity from a commercial continuous spray bottle had a coefficient of variation of 16% to 17% for ten attempts.

### Example 5

Formulation Stability—Benzocaine

Spray cans of the benzocaine formulation of Example 2 as packaged in Example 3 were held at room temperature and at 40° C. and sampled periodically. The original concentrate was also retained at room temperature and sampled periodically. The contents were evaluated by HPLC (e.g. Thermo Separation Products AS300, UV150, Waters 510, Spectraphysics Chromjet CH1, or equivalent.) The column was a Luna 5 micron C18 column, 4.6×150 mm (Part no. 00F-4252-E0). The mobile phase was methanol/pH 7.0 buffer (55:45), flow rate 1.0 to 1.5 ml/min., at ambient temperature, with measurement at 310 nm. The injection volume was 20 microliters, and the diluent was 92% methanol—8% water. Run time was typically about 30 minutes. In this system, benzocaine typically eluted at 3.5 minutes; tetracaine at 11 minutes; and butamben at 22 minutes.

It was found that the apparent concentration of benzocaine in the concentrate did not vary significantly or with any trend over a period of 4.5 months, with observed values of 97.3% of control at t=0, and values of 99.30%, 97.90%, 99.06%, 99.60%, and 98.70% at 0.07, 2.23, 3.63, 4.57 and 5.47 months, respectively. The value in the room temperature cans was 98.6 to start and 98.2 at 4.23 months. The value in the 40° C. cans was 99.13% at start and 97.9% at 4.23 months. The product is expected to be stable at room temperature for at least a year.

### Example 6

Sormulation stability—Anesthetic Combination

The drug concentrate of example 1 was aged at room temperature in the presence of air. The results are shown in Table 1. The sealed aerosol cans of example 3 containing the concentrate of Example 1 were aged at room temperature. The results are shown in Table 2. A formulation comparable to Example 1 but containing only tetracaine (no benzocaine or butamben) was aged at room temperature in the presence of air. The results are shown in Table 3. There is a clear loss of tetracaine in the multi-agent concentrate and in the combined-anesthetic can, but no similar loss of tetracaine in the tetracaine-only formulations. There is no apparent loss of benzocaine or butamben.

TABLE 1
Stability of Tetracaine/Benzocaine/Butamben Formulation
(Example 1) Aged in the Presence of Air

| Age | | | | % Tetracaine/ |
|---|---|---|---|---|
| (months) | Benzocaine | Butamben | Tetracaine | % Benzocaine |
| 0.00 | 102.52 | 101.08 | 102.55 | 1.00 |
| 0.93 | 99.85 | 99.58 | 96.99 | 0.97 |
| 2.50 | 98.50 | 98.90 | 96.60 | 0.98 |
| 3.70⁽¹⁾ | 101.40 | 99.40 | 93.70 | 0.92 |
| 3.70⁽²⁾ | 101.70 | 100.20 | 96.10 | 0.94 |
| 4.93 | 98.80 | 99.00 | 92.50 | 0.94 |
| 5.83 | 98.54 | 98.85 | 92.22 | 0.94 |

⁽¹⁾Measured at the top of the can.
⁽²⁾Measured at the bottom of the can.

TABLE 2
Stability of Tetracaine/Benzocaine/Butamben (Example 1)
Prepared and Packaged in an Aerosol Can

| Age | | | | % Tetracaine/ |
|---|---|---|---|---|
| (months) | Benzocaine | Butamben | Tetracaine | % Benzocaine |
| 0.00 | 99.13 | 98.52 | 97.60 | 0.98 |
| 4.60⁽¹⁾ | 97.81 | 97.95 | 90.41 | 0.92 |
| 4.60⁽²⁾ | 96.57 | 96.27 | 91.30 | 0.95 |

⁽¹⁾Measured at room temperature.
⁽²⁾Measured at 40°.

TABLE 3
Stability of Tetracaine-Only Formulation

| Age (months) | % Tetracaine |
|---|---|
| 0.00 | 98.50 |
| 0.23 | 102.00 |
| 0.67 | 100.90 |
| 0.93 | 100.68 |

It was found that preparation of the formulation of Example 1 in the absence of oxygen, and its loading into aerosol cans in the absence of oxygen, prevented the loss of tetracaine on storage. It appears that a specific interaction of tetracaine with oxygen occurs in the presence of certain catalytic materials, which include benzocaine and butamben, leading to the loss of tetracaine and the appearance of a possible degradation product which is detected by chromatography. This unknown material appeared in the formulations in which tetracaine was degraded, eluting at about five minutes.

Unless defined otherwise, all technical and scientific terms used herein have the same meanings as commonly understood by one of skill in the art to which the disclosed invention belongs. Publications cited herein and the material for which they are cited are specifically incorporated by reference. Those skilled in the art will recognize, or be able to ascertain using no more than routine experimentation, equivalents to the specific embodiments of the invention described herein. Such equivalents are intended to be encompassed by the following claims.

1. A topical liquid aerosol formulation for accurate metered dose delivery comprising:
(a) a concentrate comprising a local anesthetic in a non-alcohol solvent; and
(b) a hydrofluorocarbon (HFC) propellant.

2. The formulation of claim 1 wherein the concentration of the non-alcohol solvent in the concentrate is between about 75% and 85% by weight of the formulation.

3. The formulation of claim 1 wherein the non-alcohol solvent is a water-soluble polyol.

4. The formulation of claim 3 wherein the water-soluble polyol is selected from the group consisting of ethylene glycol, propylene glycol, glycerol, diethylene glycol, dipropylene glycol, oligoalkylene glycols, liquid polyalkylene glycols, and combinations thereof.

5. The formulation of claim 4 wherein the water-soluble polyol is dipropylene glycol.

6. The formulation of claim 1 wherein the concentration of the local anesthetic in the concentrate is between about 15% and 25% by weight.

7. The formulation of claim 1 wherein the local anesthetic is selected from the group consisting of lidocaine, prilocaine, bupivacaine, levo-bupivacaine, ropivacaine, mepivacaine, procaine, chloroprocaine, propoxycaine, hexylcaine, tetracaine, cyclomethycaine, benoxinate, butacaine, proparacaine, butamben, diperodon, phenacaine, falicaine, dyclonine, pramoxine, dimethisoquien, benzocaine, amethocaine, dibucaine, ketocaine, propanocaine, propipocaine, and combinations thereof.

8. The formulation of claim 7 comprising benzocaine, tetracaine, and butylaminobenzoate.

9. The formulation of claim 8 wherein the concentration of benzocaine in the concentrate is 14% by weight.

10. The formulation of claim 8 wherein the concentration of tetracaine in the concentrate is 2% by weight.

11. The formulation of claim 8 wherein the concentration of butylaminobenzoate in the concentrate is 2% by weight.

12. The formulation of claim 1 further comprising an excipient in the concentrate in a concentration of between about 0.5% and 3% by weight.

13. The formulation of claim 1 further comprising an excipient selected from the group consisting of flavoring agents and preservatives and combinations thereof.

14. The formulation of claim 13 wherein the preservative is a combination of benzalkonium chloride and cetyldimethylammonium bromide.

15. The formulation of claim 1 wherein the hydrofluorocarbon propellant is selected from the group consisting of 1,1,1,2-tetrafluoroethane and 1,1,1,2,3,3,3-heptafluoropropane and combinations thereof.

16. The formulation of claim 1 wherein the concentration of the HFC propellant is between about 35% and 65% by weight of the final formulation.

17. The formulation of claim 16 wherein the concentration of the HFC propellant is between about 45% and 55% by weight of the final formulation.

18. The formulation of claim 1 wherein the formulation is substantially free of oxygen.

19. The formulation of claim 18 wherein the oxygen is removed by process selected from the group consisting of purging the concentrate with an inert gas, cold filling the hydrofluorocarbon, preparing the formulation under vacuum, treatment with antioxidants, and combinations thereof.

20. The formulation of claim 19 wherein the inert gas is selected from the group consisting of nitrogen and argon.

21. A method of using the formulation of claim 1 for accurate metered dose delivery to a surface of a human or animal, the method comprising:
(a) providing a pressurizable container;
(b) placing a mixture comprising a local anesthetic dissolved in a non-alcohol solvent into the container;
(c) installing a metering valve for release of a controlled amount of the local anesthetic from the container at each activation of the valve;
(d) manipulating the container to form a pressure-tight seal; and
(e) charging the sealed container with a hydrofluorocarbon propellant.

22. A method for making the formulation of claim 1 for accurate metered dose delivery to a surface of a human or animal, the method comprising:
(a) dissolving the local anesthetic in a non-alcohol solvent to make a concentrate;
(b) placing the concentrate in a pressurizable container;
(c) sealing the pressurizable container; and
(d) charging the container with a hydrofluorocarbon propellant.

23. A method of stabilizing a formulation comprising tetracaine and a second local anesthetic, during storage, the method comprising rendering the formulation substantially free of oxygen.

24. The method of claim 23 wherein the oxygen is removed by a process selected from the group consisting of purging the concentrate with an inert gas, cold filling the hydrofluorocarbon, preparing the formulation under vacuum, treatment with antioxidants, and combinations thereof.

25. The method of claim 23 wherein the second local anesthetic is benzocaine.
